(19) United States
(12) Patent Application Publication
(10) Pub. No.: US 2003/0167061 A1
(43) Pub. Date: Sep. 4, 2003

(54) Medical device for stereotaxis and patient positioning

(76) Inventors: Wolfgang Schlegel, Heidelberg (DE); Otto Pastyr, Leimen (DE); Gernot Echner, Wiesenbach (DE); Volker Sturm, Wiesloch-Schatthausen (DE)

Correspondence Address: Kohler Schnid + Partner, Ruppmannstr. 27, D-70565 Stuttgart, DE

(21) Appl. No.: 10/311,877

(22) PCT Filed: Jun. 28, 2001
(86) PCT No.: PCT/EP01/07440

(30) Foreign Application Priority Data
Jul. 1, 2000 (DE) 100322034

(51) Int. Cl. A61B 19/00

(52) U.S. Cl. 606/130

(57) ABSTRACT

The invention relates to a medical device having a mechanism (1) for positioning a devise (4) by means of an arm (7), said arm consisting of at least three members (8, 9, 10), wherein the mechanism (1) has at least five adjustment axes (11, 12, 13, 14, 15, 16), fine adjustment and locking devices (17) being assigned thereto. The medical device is configured in such a way that it can be used universally, especially as a device for stereotaxis that can be placed anywhere and that does not interfere with other medical measures and also as a position determining device that can be placed anywhere for determining the position of a patient (29). This is achieved in that the mechanism (1) has a base part (5) that can be fixed in any given location in the region of the patient (29) in such a way that the device (4) can reach all given target points (2) in or on the patient (29) in practically any direction (3).

### DESCRIPTION

The invention concerns a medical device having a mechanism for positioning an instrument using an arm with at least three members, wherein the mechanism has at least five adjustment axes with associated fine adjustment and locking devices.

Medical devices of this type are used in stereotaxis for different diagnostic and therapeutical purposes such as taking tissue samples, evacuation of haematomas, aspiration of cysts, targeted laser use or implantations for many interventional radiological applications in computer tomography, magnetic resonance tomography, positron emission tomography etc. to e.g. introduce radioactive implantations for the treatment of cancer. The target point and probe direction are thereby determined through diagnosis via a localization system e.g. as mentioned above. This determination is done by a physician and is often aided through calculation by a planning program. The target goal may be removal of a tumor tissue sample and the probe direction towards this target point is determined such that the advance to the target point is as advantageous as possible, i.e. avoids sensitive organs. These operations require high precision and safety and are usually carried out through positioning and guiding systems for probes, with the direction of introduction and often the target point being mechanically defined to obtain the required accuracy and safety.

A device of the above-mentioned type for stereotaxis is disclosed in U.S. Pat. No. 6,035,228. This stereotaxis device is disposed on an imaging means mounted to a rail extending transverse to the patient and can therefore guide instruments to a target point using data from the imaging means. The proposed imaging means is a tube-like X-ray device into which the patient is introduced which, since it surrounds the patient, permits image acquisition from all sides. This stereotaxis device has only limited uses for the above-mentioned purpose, since it cannot reach all target points in the human body, in any direction, and in an unlimited fashion due to the fixed association with the imaging device which, in turn, can only be displaced along the rail. Since the arm of this device has two sequential axes of rotation which are oriented in the same direction, the degrees of freedom for possible movement are limited, which speaks against universal applicability. The device is designed and determined for use with surgical instruments on the basis of imaging device data, mainly for taking tissue samples. The rigid connection to the imaging device which surrounds the patient like a portal, prevents this conventional stereotaxis device from assuming any position with respect to the patient. For this reason, it cannot be used for operations, at least not for major operations, since the portal-like device does not permit access to the patient from all sides.

Moreover, this known device is not intended to have other principal applications. In addition to approaching target points for treatment or diagnosis, the position of a patient must also be measured, e.g. to avoid repeated use of an imaging device for recurrent treatments to simplify the procedure and also avoid damage to the patient caused e.g. by frequent, repeated use of imaging, e.g. with an X-ray device.

WO 00/33755 discloses a surgical instrument holder having a moveable, adjustable arm. The arm has an associate stand having a height adjustment mechanism for the arm. The arm has a plurality of members, but all of the first three axes have a vertical orientation. The degrees of freedom for adjustment are therefore highly limited and possible uses are essentially limited to the holding of instruments.

WO 99/37220 discloses an orthopedic instrument for amputations having arms for grasping limbs and holding tools. The arms also have a plurality of links coupled together and having a same rotational axis orientation. This also results in a likewise limited scope of adjustability and consequently limited applications.

It is the underlying purpose of the present invention to propose a device of the above-mentioned type which can be universally used, in particular as a device for stereotaxis, which can be placed at any location and which presents minimal impediment to further medical procedures as well as to present a position determining device for determining the position of a patient which can also be disposed at any desired location.

This object is achieved in accordance with the invention in that the mechanism of the device comprises a support part which can be mounted at any location proximate the patient such that an instrument can reach any target point in or on the patient from nearly any direction.

Mechanisms which are based on a construction principle comparable to that of the inventive device are known as handling or measuring robots. However, such robots are not suited in their present form for medical operations without substantial technical effort to ensure that the robot satisfies the safety requirements of medical product law. This technical effort would render such a robot too expensive for general use. Moreover, the design of such robots is based on different requirements which are less stringent with regard to safety and which are substantially more demanding with respect to motion coordination. Such robots must carry out all types of motion to perform e.g. assembly processes. The present medical field of use is limited to fixing an instrument with respect to a direction of motion and to orientation towards a target point on or in the body to exactly and safely reach this target point from a position outside of the body through linear displacement, wherein the patient or the relevant part of the body of the patient must, of course, be stationary. Such fixing is known e.g. from radiation therapy. For the above-mentioned reasons, the inventive device does not require extremely demanding three-dimensional motion control. Rather, it is sufficient to sequentially direct the adjustment axes to allow a physician to introduce an instrument, e.g. a probe into the body to a predetermined target point after the instrument is positioned and its target direction and position are checked and adjusted to perform the required action at the target point.

In a corresponding fashion, it is sufficient to approach a position of a target point which is to be measured from one direction. In this case as well, the target point, e.g. a marking or a calibration point, must be simply reached and the path of approach is usually not important when the target point is located on or outside of the patient. In this case as well, three-dimensional control of the coordinated motion sequence is not required.

If the inventive device is provided with drives and can be operated in a computer-controlled fashion, its operation is always limited to a sequence of settings which can be adjusted one after the other or independently of one another and does not require simultaneous coordinated motion of several joints. The settings can be calculated beforehand and such calculation is substantially less demanding than coordination of several simultaneous adjustment motions.

There is one very essential aspect with regard to safety: The settings can be prepared and thoroughly checked without any risk to the patient from the motor-driven components. The adjustment motion during treatment is limited to advance of an instrument, e.g. a probe, to the target point along the set and checked adjustment path. The adjustment motion that affects the human body is one-dimensional and not three-dimensional as required by a robot. This motion can be monitored and carried out with little technical effort and high safety standards. The settings can be effected with very high precision and without expensive control measures. The predetermined position of the probe can be obtained and fixed in accordance with the invention through fine adjustment and locking devices associated with the adjustment axes.

An embodiment of the inventive device having a support part which can be placed anywhere in the proximity of the patient and which is easy to move, permits use of the inventive medical device not only universally for the most differing operations and examinations in stereotaxis, but also for additional applications such as patient positioning. In contrast to stereotaxis where a predetermined target point is approached, in this case, one or more target points on the patient, e.g. markings can be addressed to determine the position of the patient. From the position determination, either the position of the patient can be changed or preferably the location to be treated or examined is calculated from the assumed position. Treatment is then started and may be any kind of treatment, radiation procedure, or examination which can be carried out with the inventive device. The invention is particularly advantageous, since the device can be used both for positioning the patient as well as for treatment. In the first case, the location of the patient is determined, corrected, or used in calculations and also verified. The instrument for treatment or examination can then be mounted to the arm of the device and the operation can be carried out.

In this fashion, the use of the inventive device—if necessary with the corresponding relevant designs—avoids new imaging for each recurrent treatment or examination, as is required by U.S. Pat. No. 6,035,228. The particular advantage of the inventive object consists in that it can be universally used for many procedures, in particular for subsequent work steps such as positioning and, if necessary, further examination and treatment.

The invention can be advantageously used for the treatment, diagnosis and position determination within the entire body and an instrument can reach almost any point in almost any direction. Such an instrument can include all devices used for surgery, diagnosis or position determination. In addition to the above-mentioned applications, position determination using devices such as samplers or many others is also possible. This is advantageous for operations or examinations since target points can be addressed from all directions. It is therefore possible to select the gentlest direction of insertion for the patient in stereotactic operations. Target points may be in the body, on the body or on a part which is rigidly connected to the body depending on application of the device. Since any location is possible for the mechanism, the location can be selected such that the mechanism is positioned while maintaining an optimum insertion direction such that it does not obstruct further procedures. The mechanism can be adjusted with high precision and probes or other instruments of any type can be used, even those which serve for position determination in patient positioning—a use which will be described further below.

It is important that the support part be easily moveable to all possible locations and then be fixed in the desired position. The location must not be on the floor, and mountings to other objects or the ceiling are also possible. It is important that the positioning be highly variable. For rigid fixing, the support part may comprise a means for suppressing undesired change of location. It may be fixed to the floor in the region of the patient. The support is advantageously sized to provide adequate stability without occupying space which is needed for other medical or further devices. Small position changes with high stability can be achieved e.g. when the support part has rollers and an associated means for suppressing rolling motion, e.g. brakes. Other means for fixing the device are feasible such as extendable supports, retractable rollers, magnetic holders or the like.

One further possibility of fixing to a location is effected through connection of the support part to a part which is rigidly mounted to the patient. The support part may be connected e.g. to a fixing device. If the device is relatively small for operations in the head region, the support part can be mounted to a fixing ring for the head. Mounting to all kinds of fixing devices is possible as is connection to the patient table, in particular if same is provided with fixing devices. The positioning possibilities should also be variable in this case.

One substantial advantage of the invention compared to the prior art of U.S. Pat. No. 6,035,228 is that the device is not connected to the tomography device, but can be rigidly connected to the patient to avoid constant use of a tomography device for continuous position detection. This is particularly important with regard to harmful X-rays and additionally permits procedures with which a tomography device would be disruptive. In this regard, fixed connection to the patient is particularly advantageous as is provided not via the floor but via a part to which he/she is fixed, e.g. a head fixing ring or a fixing means on the patient bed. The patient and device can then be moved between the operating region and an imaging means (X-ray device, magnetic resonance tomography device) to permit interruption of an operation and continuation thereof following verification. One can e.g. determine whether a placed biopsy needle is oriented exactly in the direction of the target point or requires correction. A precision is thereby achieved which has not been previously possible, even for operations which cannot or should not be controlled by continuous imaging.

The mechanism of the inventive device preferably has a further sixth adjustment axis which also has associated fine adjustment and locking devices. In this fashion, the device can be used even more universally and there are no limitations for reaching any point from any direction.

The front member is preferably provided with a holder for receiving the most differing of instruments to permit use for all possible diagnostic and therapeutical procedures as well as position determination. A probe can e.g. be introduced in the precisely linear manner required therefor. The front member of the arm can thereby have a guide which can be directed towards a target point for straight introduction of an instrument. The front element may be telescopic. If a holder is provided with such a guide, it can extend parallel or at an angle with respect to the front member. The latter is advantageous in that the device does not obstruct access when the physician wants to get as close as possible to the treatment region. There are further possibilities for linear motion of an instrument, e.g. the arm may be provided with a further adjustment axis which is coupled to two adjustment axes whose elements can be moved in the same or parallel planes such that the adjustment axes perform opposite angle adjustments which mutually cancel and simultaneously perform correction to maintain the position of the means in the direction towards the target point. This mechanism prevents angle shifts as well as parallel shifts of the probe while it is moved to a target point. Any embodiment is possible which guarantees straight introduction of e.g. a probe with great precision and safety. In a corresponding fashion, any design is possible which permits the position of a target point serving for position detection and correction to be determined through positioning and angle adjustment of the arm.

The angle positions for the adjustment axes required for the positioning can be determined in any appropriate manner. The detection of the support position and the angle settings of the members required for position detection of a target point can be performed in different ways. They can be detected externally e.g. by a digital camera with corresponding image processing or the adjustment axes can be provided with goniometers. Moreover, a means can be provided for determining the coordinates of any locations of the support, on the floor, on the ceiling or on a location designed for mounting, relative to a part which is fixed with respect to the patient. Detection of location can include a means in the floor or on the ceiling, a digital camera or means further described below.

For practical reasons and time efficiency, the device can be advantageously connected to a computer and comprise a program for the computer which associates a freely selectable target point with a freely selectable location of the support via the angle settings of the adjustment axes such that the one can be calculated on the basis of the coordinates of the other. This permits universal use such that the program calculates the angle settings of the adjustment axes for obtaining a target point for locations of the support. The determined values can e.g. be set on corresponding adjusting dials of the fine adjustments of the adjustment axes. The adjustment axes can be provided with goniometers thereby obtaining improved display accuracy and therefore improved adjustment accuracy of the angles.

The other principal application of the invention is to determine the position of the patient. Towards this end, the device may comprise a computer program which calculates the position of a target point addressed by a probe on the basis of the location of the support and the angle adjustments of the adjustment axes. Through the data obtained in this fashion, the patient can be positioned for treatment or examination, wherein “electronic positioning” is preferred in that the data for the planned treatment, e.g. radiation or stereotaxis is calculated as a coordinate transformation, based on the position of the patient.

To determine the positions of the support, the arm is or can be provided with a position determination device. The front end of the arm can accommodate e.g. a sampler, e.g. mounted in a probe holder. The position determination device addresses reference points, e.g. of a localizing system, to determine the position of the support at the target location and then calculates and sets the angle values of the adjustment axes relative to the selected location as is required for the desired probe positioning for motion towards a target. In the same fashion, targets on the patient can also be addressed to determine, check or correct his/her position.

In a further development, the device has a computer program which determines the position of freely selected locations of the support through three calibration points defined as target points. In this fashion, the location of the support can be determined analogous to the target point determination through defined location of the support or vice versa via addressing of calibration points. If the calibration points have a defined relation to the patient, the coordinates of the support and target point can be associated such that each location of the support easily reaches the target point. If necessary, the location is changed to permit travel along a desired introduction path. Position errors of the patient can also be taken into consideration through association of the coordinate systems to thereby eliminate errors.

To determine the position of the patient after determination of the location of the support using calibration points, the program is suitably designed to check the position of at least one addressed target from the angle positions of the adjustment axes. Towards this end, several markings on the patient which serve as target points or markings with fixed relation to the patient may serve for determining and for fixing his/her position. The markings can be disposed on the skin of the patient, on recepticals for receiving body parts or on another fixing device such as a fixing ring for the head or to a denture. The fact that the support can be arbitrarily disposed in the region of the patient is advantageous, since markings can be detected at any location.

A program for examinations and operations is suitably designed to determine the path of introduction until a target point in the body of a patient is reached to assure improved accuracy. It is then suitable to provide a measuring device for the path of introduction which has an associated, correspondingly precise display. Advantageously, the device has a computer which is loaded with a program to determine the position of at least one target point from the location of the support or vice versa.

The device comprises at least the above-mentioned mechanism and advantageously one of the above-mentioned programs. A conventional computer can be used or it is possible to equip the device with a computer which is loaded with a predetermined program and is optionally modified for the present invention. It is also possible to provide a special computer for the above-mentioned purposes.

For rapid processing, the goniometers and the position determination device are connected to a computer which determines the data for location of the support, setting of the mechanism, and the target point. This may be used to reach a predetermined target point or determine the position of an addressed target point. In the first case, the program can be designed such that it directly converts the data or informs an operator about the angle settings to be taken. The information may be communicated when the program is designed such that a computer shows the data which is required to reach the target point or other important data on a display means, usually a screen. The actual settings following adjustment are advantageously detected by the goniometers and displayed by the display device.

In a further development of the invention, the device is provided with a navigation system to detect the position of an addressed target which is displayed on the display means. It may include e.g. a camera system or sampler system for detecting the position of calibration points, markings or the instrument itself which is mounted to the arm. However, the device can also be linked to a stereotactic localization system which detects the position of the instrument and the target point. Although any localization system is possible, one is preferably selected which can be operated without danger to the patient for a prolonged period of time such as e.g. a magnetic resonance tomograph. The position of the instrument relative to the target point can be displayed on a screen thereby providing exact control for the physician performing the procedure.

In the above-mentioned embodiments, the settings were performed manually, without drives. In accordance with the invention, the settings can be made sequentially when stereotaxis is used. This does, however, not preclude the adjustment axes from having drives and a computer can be programmed for automatic adjustment of the required angle settings. This also maintains the inventive, advantageous high safety with reasonable calculating and control effort, since a position is addressed via settings which need not be time-coordinated. The setting can be checked before the actual operation on the body. The operation takes place in one dimension and can be carried out with little effort, high precision and high safety. The instrument, e.g. a probe, can be introduced by hand, if necessary via an exactly adjustable translation mechanism or the instrument can be guided along the path of insertion to the target point via a further drive.

To complement such drives, the invention proposes designing the program for computer-control of the drives and if necessary, of a further drive associated with the instrument for carrying out a diagnostic or therapeutic procedure. In this case, the operation is automatic and the task of the doctor is previous determination of the data and monitoring of the operation. The computer is suitably provided with an input means for controlling the operation.

The locking devices can be designed such that they must be manually operated when the fine adjustments are obtained. Alternatively, the locking devices automatically lock the adjustment axes, when the respective calculated desired position is achieved. This function control can be integrated in a computer program which receives the corresponding actual data via the goniometer and compares them with the desired data.

To further increase safety, the device for stereotactic use is provided with a device for issuing an alarm when one of the settings of the mechanism is no longer in the desired position.

If the device for stereotaxis is to be used in connection with detection via a magnetic resonance tomograph, at least the front regions of the arm are advantageously made from electrically non-conducting and non-magnetizable materials. Fiber reinforced materials or plastic materials with glass fiber reinforcement are suitable.

The length of the arm can be dimensioned to reach target points throughout the entire human body. However, the length of the arm can also be dimensioned to reach points on a partial region of the human body, e.g. the head. In this case, the arm may be considerably shorter. The device can then be used for a limited region and precision is considerably higher due to the shorter length of the arm. This may be important, in particular, for use in the head region. The inventive advantages over prior art mentioned above are still maintained, since the inventive design still permits all insertion directions in the head region and can reach any marking or other measuring point, not merely those in the upper skull region.

To obtain a high safety standard, the device can preferably have an associated measuring phantom comprising a test target point which can be brought into the position of the actual target point, wherein the position determination device detects when the test target point is reached by the relevant instrument. The sampler can e.g. be placed at the location of the relevant instrument and thereby serve as a position determination device. For probes, the relevant probe part is that part of the probe which is to reach the target point and treat same. Such measuring phantoms can be designed in any appropriate fashion. The above-mentioned prior art shows an example thereof. Such a measuring phantom adjusts a device which then serves for patient positioning or verification thereof.

The support part of the device can also be mounted to the ceiling above the patient. It has an appropriate device for positioning, e.g. a cross slide mechanism or staging.

The invention is explained below with reference to an embodiment shown in the drawing.

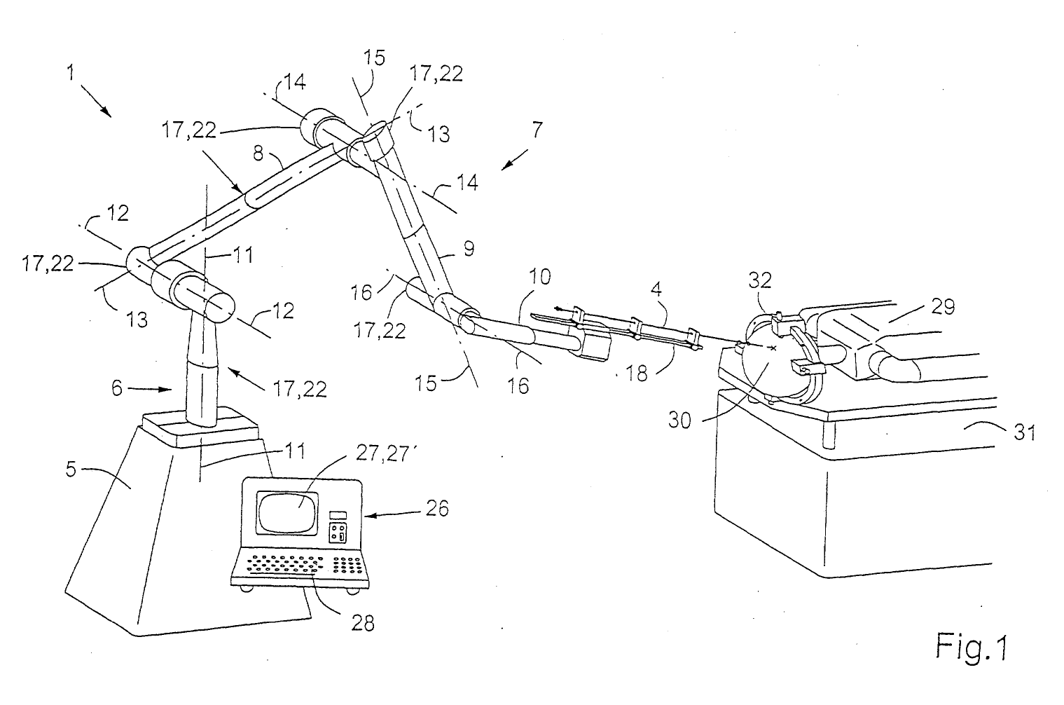

FIG. 1 shows an embodiment of the invention;

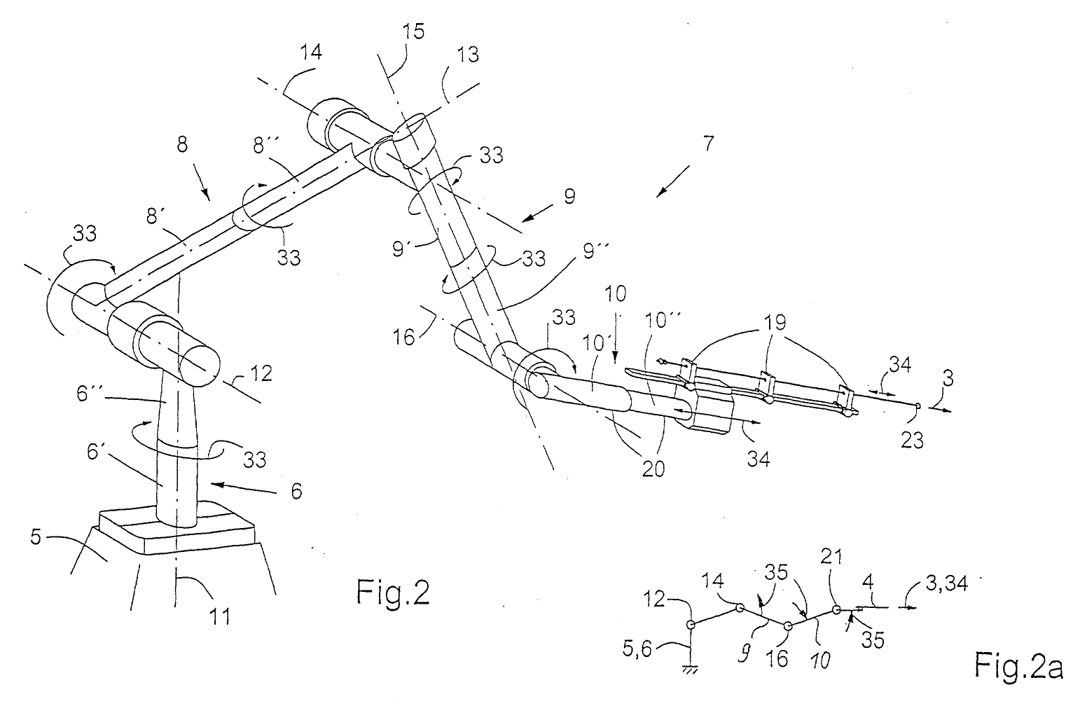

FIG. 2 shows the mechanism of the embodiment of FIG. 1;

FIG. 2a shows a schematic diagram of an alternative design of the mechanism; and

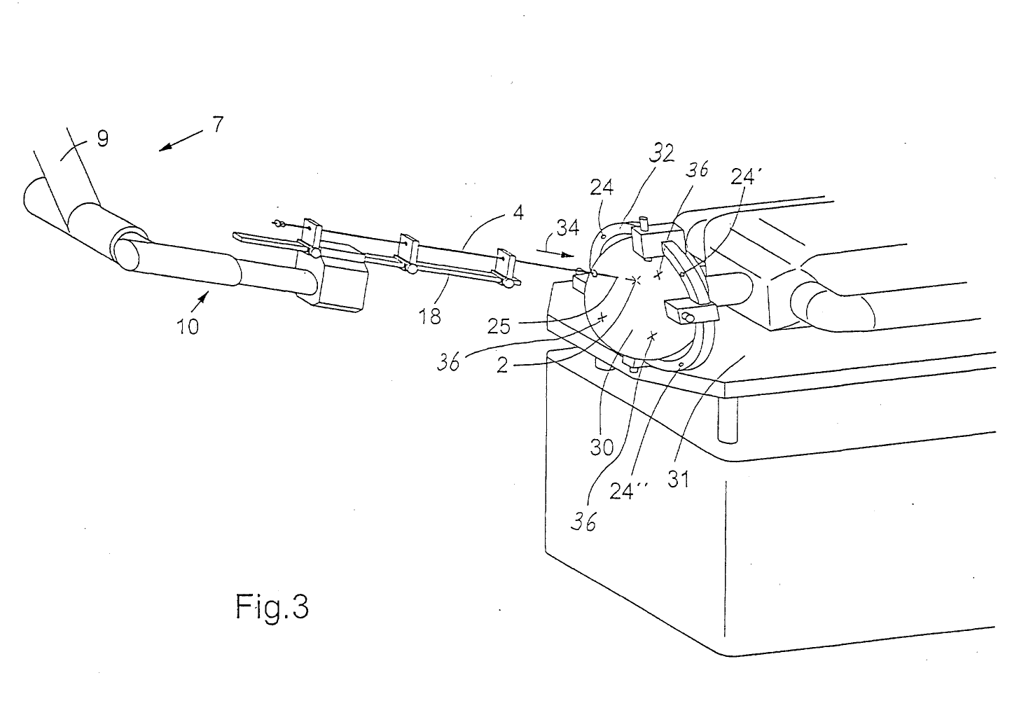

FIG. 3 shows an enlarged view of the insertion region.

FIGS. 1, 2 and 3 show the same embodiment of the invention, wherein the same reference numerals indicate identical parts. For reasons of clarity, not all reference numerals are inserted in all illustrations. FIG. 1 shows an overall view of the embodiment of the invention with the mechanism 1 comprising the arm 7, wherein an operation is effected on the head 30 of a person using a probe 4. The arm 7 is shown in an enlarged scale in FIG. 2 with additional details included for explanatory reasons. The operation itself is shown in an enlarged scale in FIG. 3.

The mechanism 1 is designed such that a probe 4 is positioned on a human body 29, e.g. a head 30 such that the probe 4 can be brought to a target point 2 via linear displacement thereof. Towards this end, a support 5, 6 is provided to carry the arm 7 and comprises at least three members 8, 9 and 10. The arm 7 has at least five adjustment axes 11, 12, 13, 14, 15, 16 and fine adjustment and locking devices 17 are associated with these adjustment axes 11, 12, 13, 14, 15, 16. The arm 7 is hinged to a support part 5 via a rotatable support part 6 and fixed to the floor. The support 5, 6 is dimensioned in size and weight such that it can be placed anywhere in the region of the patient 29 and does not obstruct any medical procedures or block access for other instruments. It must nevertheless be absolutely stable (see the above embodiments).

The support part 6 consists of two mutually rotatable parts 6′ and 6″ which permit a rotary motion 33. This pivots the arm 7 within a room. A member 8 of the arm 7 is coupled to the support part 6, wherein the coupling has a further adjustment axis 12 designed as an axis of rotation which also permits a rotary motion 33 perpendicular to the first rotary motion 33. This adjustment axis 12, like all other adjustment axes 13, 14, 15, 16 can perform the respectively illustrated rotary motions 33 and all are provided with fine adjustment and locking devices 17. The member 8 mounted to the support 5, 6 also comprises two rotary parts 8′ and 8″ and the joining intermediate member 9 has two rotatable parts 9′ and 9″. The coupling of the intermediate member 9 to the member 8 also includes an adjustment axis 14 constructed as an axis of rotation as is the coupling of the front member 10 to the intermediate member 9 via the adjustment axis 16. In contrast thereto, the front member 10 can comprise two telescopically displaceable parts 1′ and 1″ which form a guide 20 for a linear motion 34 to introduce a probe 4. Alternatively, a probe holder 18 can also have a guide 19 for the same purpose.

Instead of a probe 4, another instrument 4 can also be mounted to the arm 7 e.g. a sampler for detecting markings 36 on the patient 29 e.g. on the head 30 (see FIG. 3). When the markings 36 are addressed and their position detected, the exact position of the patient 29 or of a body part, e.g. head 30, can be detected and the position and preferably the calculation corrected to precisely address a target point 2.

The probe 4 must be appropriately positioned in front of the body 29 to move the probe 4 to the insertion position from which it moves, after penetration of the body surface, along the path of introduction 25 to a target point 2 along the introduction direction 3. This position is defined by the target point 2 and direction 3. Towards this end, the mechanism 1 with support 5 is positioned such that it minimally obstructs further medical procedures while following, from a medical point of view, an optimum path of introduction 25. When the support 5 is positioned, a sampler 23 is inserted in the probe holder 18 and the arm 7 is gauged. Towards this end, a fixing device 32 for the human body 29, herein e.g. head 30, carries three calibration points 24, 24′, 24″, which are addressed by the sampler 23. The sampler 23 issues a signal at the corresponding positions and the goniometers associated with the adjustment axes 11 through 16 transfer the associated angle positions to a computer 26 which can thereby determine the exact position of the mechanism 1 and of the support 5, 6 and arm 7. If such a fixing device 32 remains on the patient 29 between image acquisition and operation, the calibration points 24, 24′, 24″ can serve as markings 36 for patient positioning. Alternatively, the position of the fixing device 32 can be determined by detecting the calibration points 24, 24′, 24″ and comparison with the position of additional markings 36.

In a stereotactic operation, an operator uses an entering means 28 to enter the previously determined data of the target point 2 as well as the introduction direction 3 which includes the medically most favorable introduction path 25. The computer 26 then calculates, using an appropriate program, the required angle positions which must be set at the adjustment axes 11 through 16. The arm 7 is thereby brought into the correct position in space via the adjustment axis 11 and the distance is set via the adjustment axes 12, 14 and 16. The corresponding angle in space is addressed via the adjustment axes 13, 15 and 16.

Although this is a complicated adjustment process, it can be performed without time loss, since the computer 26 determines the adjustment data and displays it on a display means 27, e.g. a screen 27′, wherein an operator can subsequently address these settings on adjustment axes 11 to 16.

All adjustment axes 11 through 16 of the arm 7 are suitably provided with goniometers 22 which are connected to the computer 26 thereby permitting exact display of the actual positions and preferably also of the desired positions on the screen 27. Preferably, the fine adjustment and locking devices 17 are automatically locked by the computer 26 when the desired position is reached. When all settings are obtained, the sampler 23, if used as position determining device, is replaced by a probe 4 and if necessary the position is precisely checked before or after this replacement.

The procedure shown in FIG. 3 can then be performed. The physician can guide the probe 4 through the body surface to the target point 2, if necessary following drilling through the top of the skull, and starts treatment or diagnosis. The respective position of the probe 4 can be detected and shown on the screen 27′ to permit exact monitoring. When used as positioning device, points such as markings 36 or calibration points 24, 24′, 24″ must be addressed to determine their coordinates thereby also determining the position of the patient 29 or of a body part 30 and a target point 2 can then be precisely addressed.

The embodiment of the invention in accordance with FIGS. 1, 2 and 3 is of course only an exemplary embodiment. FIG. 2a shows that e.g. the linear motion 34 in direction 3 can also be obtained by disposing three adjustment axes 14, 16, 21 in one plane which cooperate such that the probe 4 exerts a linear motion 34. These adjustment motions are shown by the arrows 35.

Clearly, many additional embodiments are possible. The adjustment axes 11 through 16 can also be arranged in a different fashion. The only requirement is that a variety of possible adjustments is guaranteed which permit exact positioning of an instrument in space with orientation in direction 3 aiming towards a target point 2.

The illustrations show only one embodiment. The support 5 can also be a mounting plate with which a relatively small arm 7 can be fixed directly on a fixing device 32, e.g. a head holding ring to reach a target point 2 in the head 30. Further embodiments of the support part 5 comprise means (not further illustrated) for preventing undesired location changes. The further embodiments which are not shown are disclosed in the general description of the invention.

The device can also be exclusively used as a positioning and verification device to check the exact position of a target point 2 for carrying out another procedure, e.g. irradiation, and correct the position, if necessary, or to carry out a procedure indicated by the determined location.

List of Reference Numerals

1 mechanism

2 target point

3 direction

4 instrument, e.g. probe (for medical operation; examination or position measurement)

5, 6 support

5 support part for fixing on the floor

6, 6′, 6″ support part for coupling the arm

6′, 6″ rotatable parts of the support part for coupling the arm

7 arm

8, 9, 10 members of the arm

8 member disposed on the support

8′, 8″ rotatable part of the member disposed on the support

9 intermediate member

9′, 9″ mutually rotatable parts of the intermediate member

10 front member

10′, 10″ mutually telescopically displaceable parts of the front member

11 through 16 adjustment axes

11 adjustment axis of the rotatable parts of the support part for coupling of the arm

12 adjustment axis between the support part for coupling the arm and arm

13 adjustment axis of the rotatable parts of the member of the arm disposed on the support

14 adjustment axis between the member disposed on the support and the intermediate member

15 adjustment axis of the rotatable parts of the intermediate member

16 adjustment axis between intermediate member and front member

17 fine adjustment and locking devices

18 holder, e.g. probe holder

19 guide on the probe holder

20 guide on the front member of the arm

21 further adjustment axis

22 goniometer

23 sampler

24, 24′, 24″ three calibration points

25 insertion path

26 computer

27, 27′ display means

27′ screen

28 input means

29 patient (entire human body)

30 head

31 patient table

32 fixing device (e.g. head holding ring)

33 arrows: rotary motions

34 arrows: linear motion

35 arrows: adjustment motions of three axes for obtaining a straight probe motion

36 markings on the patient e.g. on the head

1. Medical device comprising a mechanism (1) for positioning an instrument (4) using an arm (7) having at least three members (8, 9, 10), wherein the mechanism (1) has at least five adjustment axes (11, 12, 13, 14, 15, 16) with associated fine adjustment and locking devices (17), characterized in that each adjustment axis (11, 12, 13, 14, 15, 16) permits a rotational motion (33) which is perpendicular to a rotational motion (33) of an adjacent adjustment axis (11, 12, 13, 14, 15 or 16) and the mechanism (1) has a support part (5) for fixing to any location in the region of the patient (29) such that the instrument (4) can reach any target point (2) in or on the patient (29) from nearly any direction (3).

2. Device according to claim 1, characterized in that the support part (5) has a means for suppressing undesired location changes.

3. Device according to claim 2, characterized in that the support part (5) can be fixed to the floor in the region of the patient (29).

4. Device according to claim 1, 2 or 3, characterized in that the support part (5) is sized to ensure sufficient stability but does not occupy any space which should be provided for medical measures or further instruments.

5. Device according to claim 3 or 4, characterized in that the support part (5) has rollers and a means for suppressing motion of the rollers.

6. Device according to claim 2, characterized in that the support part (5) can be connected to a part which is fixed relative to the patient (29).

7. Device according to claim 6, characterized in that the support part (5) can be connected to a fixing device (32).

8. Device according to claim 6, characterized in that the support part (5) can be connected to the patient table (31).

9. Device according to any one of the claims 1 through 8, characterized in that a further sixth adjustment axis (11, 12, 13, 14, 15 or 16) is provided which also has an associated fine adjustment and locking device (17).

10. Device according to any one of the claims 1 through 9, characterized in that the front member (10) is provided with a holder (18) for mounting the most differing of instruments (4).

11. Device according to claim 10, characterized in that the holder (18) has a guide (19) which can be directed towards (3) a target point (2) for linear displacement of an instrument (4).

12. Device according to claim 11, characterized in that the guide (19) of the holder (18) extends at an angle with respect to the orientation of the front member (10).

13. Device according to any one of the claims 1 through 10, characterized in that the front member (10) of the arm (7) has a guide (20) which can be directed towards (3) a target point (2) for linear displacement of an instrument (4).

14. Device according to any one of the claims 1 through 10, characterized in that a further adjustment axis (21) is provided for linear displacement of an instrument (4) which is coupled to two adjustment axes (14, 16) and whose members (9, 10) can be moved in the same or parallel planes such that the adjustment axes (21, 14, 16) perform oppositely directed angular displacements which cancel to simultaneously effect correction while maintaining the position of the instrument (4) in the direction (3) of a target point (2).

15. Device according to any one of the claims 1 through 14, characterized in that the adjustment axes (11, 12, 13, 14, 15, 16 and if necessary 21) are provided with goniometers (22).

16. Device according to any one of the claims 1 through 15, characterized in that it can be connected to a computer (26) and comprises a program for a computer (26) which associates, through the angle positions of the adjustment axes (11, 12, 13, 14, 15, 16 and optionally 21), a freely selectable target point (2) and a freely selectable location of the support (5, 6) such that one can be calculated on the basis of the known coordinates of the other.

17. Device according to claim 16, characterized in that a means is provided for determining the coordinates of any position of the support (5, 6).

18. Device according to claim 16 and 17, characterized in that it comprises a program for a computer (26) which calculates, for locations of the support (5, 6), the angle positions of the adjustment axes (11, 12, 13, 14, 15, 16 and optionally 21) for reaching a target point (2).

19. Device according to claims 16 and 17, characterized in that it comprises a program for a computer (26) which calculates the position of a target point (2) addressed by an instrument (4) on the basis of the location of the support (5, 6) and the angle positions of the adjustment axes (11, 12, 13, 14, 15, 16 and optionally 21).

20. Device according to claim 17, 18 of 19, characterized in that the arm (7) is provided with a position determination device.

21. Device according to any one of the claims 17 through 20, characterized in that it comprises a program for a computer (26) which determines the location of a freely selected position of the support (5, 6) using three calibration points (24, 24′, 24″), defined as target points (2).

22. Device according to claim 21, characterized in that the calibration points (24, 24′, 24″) have a defined relationship to the patient (29) such that the relative position of the support (5,6) with respect to the patient (29) can be determined.

23. Device according to claim 21 or 22, characterized in that the program determines the angle positions of the adjustment axes (11, 12, 13, 14, 15, 16 and optionally 21) for reaching a predetermined target point (2) after determination of the location of the support (5, 6).

24. Device according to claim 21 or 22, characterized in that the program checks the position of at least one addressed target point (2) from the angle positions of the adjustment axes (11, 12, 13, 14, 15, 16 and optionally 21) after determination of the location of the support (5, 6) using the calibration points (24, 24′, 24″).

25. Device according to claim 24, characterized in that the position of several markings (36) on the patient (29) which serve as target points (2) or of markings (36, 24, 24′, 24″) with fixed relation to the patient (29) serve for determination and/or examination of his/her position.

26. Device according to any one of the claims 18 through 23, characterized in that the program also determines the path of introduction (25) until a target point (2) in the body of a patient (29) has been reached.

27. Device according to claim 16 through 26, characterized in that it is provided with a computer (26) which is loaded with a program for determining the position of at least one target point (2) from the location of the support (5, 6) or vice versa.

28. Device according to any one of the claims 17 through 27, characterized in that the goniometers (22) and the position determination device are connected to a computer (26) which determines the data of the location of the support (5, 6), the adjustment of the mechanism (1), and data of a target point (2).

29. Device according to claim 28, characterized in that it serves to reach a predetermined target point (2).

30. Device according to claim 28, characterized in that it serves for determining the position of an addressed target point (2).

31. Device according to any one of the claims 17 through 30, characterized in that the program is designed such that a computer (26) displays the data on a display device (27, 27′).

32. Device according to claim 29 or 31, characterized in that the results of performed adjustments are detected by the goniometers (22) and displayed by the display means (27, 27′).

33. Device according to any one of the claims 1 through 32, characterized in that it is provided with a navigation system for detecting the position of an addressed target point (2) of the probe (4), which is displayed on the display means (27, 27′).

34. Device according to claim 33, characterized in that it is provided with a camera system or sampler system for detecting a target point (2) which is addressed by the instrument (4).

35. Device according to any one of the claims 1 through 34, characterized in that it is linked with a stereotactic localization system which detects the positioning of the instrument (4) towards the target point (2).

36. Device according to claim 35, characterized in that the positioning of the instrument (4) towards the target point (2) is displayed on a screen (27″).

37. Device according to any one of the claims 16 through 36, characterized in that the adjustment axes (11, 12, 13, 14, 15, 16) are provided with drives and a computer (26) can be programmed for automatic setting of the required angle positions.

38. Device according to claim 37, characterized in that the instrument (4) can be guided along the path of introduction (25) to the target point (2) via a further drive.

39. Device according to claim 38, characterized in that the program is designed such that it causes, via the computer (26), the drives, and if necessary at least one further drive associated with the instrument (4), to perform a diagnostic or therapeutic operation.

40. Device according to claim 39, characterized in that the computer (26) has an input means (28) for controlling the operation.

41. Device according to any one of the claims 16 through 40, characterized in that, when a respective calculated desired position is achieved, the fine adjustment and locking devices (17) automatically lock the adjustment axes (11 through 16).

42. Device according to any one of the claims 16 through 41, characterized in that it is provided with a device for issuing an alarm when one of the settings of the mechanism (1) is no longer in its desired position.

43. Device according to any one of the claims 1 through 42, characterized in that at least the front regions of the arm (7) are produced from electrically non-conducting and non-magnetizable materials.

44. Device according to any one of the claims 1 through 43, characterized in that the length of the arm (7) is dimensioned to reach target points (2) throughout the entire human body (29).

45. Device according to any one of the claims 1 through 43, characterized in that the length of the arm (7) is dimensioned to reach target points (2) on a partial region of the human body (29).

46. Device according to claim 45, characterized in that the length of the arm (7) is dimensioned to reach target points on the head (29).

47. Device according to any one of the claims 1 through 46, characterized in that a measuring phantom is associated therewith which has a test target point which can be brought to the position of a target point (2) and a position determination device detects when the test target point has been reached.

48. Device according to claim 47, characterized in that the instrument is a sampler (23) which serves as a position determination device.

49. Device according to any one of the claims 1 through 48, characterized in that the support part (5) can be mounted to the ceiling above the patient (29).
